(19) United States
(12) Patent Application Publication
(10) Pub. No.: US 2012/0283469 A1
(43) Pub. Date: Nov. 8, 2012

(54) INTERCONVERSION BETWEEN ISOMERIC P-MENTHANE-3-CARBOXYLIC ACIDS

(76) Inventors: Mark B. Erman, Atlantic Beach, FL (US); Gennadiy G. Kolomeyer, Jacksonville, FL (US); Joe W. Snow, Kingsland, GA (US)

(21) Appl. No.: 13/099,920

(22) Filed: May 3, 2011

(51) Int. Cl. C07C 51/42; C07C 51/43; C07C 51/44

(52) U.S. Cl. 562/400

(57) ABSTRACT

A process for interconversion between WS-1 and neo-WS-1 by heating to a temperature in a range of from 60 degrees Celsius to 250 degrees Celsius. The heating can be done in the presence of an acid catalyst. Starting from practically pure (=98%) WS-1, or mixtures of WS-1 and neo-WS-1, practically pure (=98%) neo-WS-1 can be obtained. Starting from practically pure (=98%) neo-WS-1, or mixtures of WS-1 and neo-WS-1, practically pure (=98%) WS-1 can be obtained.

### FIELD OF THE INVENTION

The invention generally relates to synthesis of isomeric p-menthane-3-carboxylic acids, and more particularly to synthesis of WS-1 and neo-WS-1 as shown in Scheme 1.

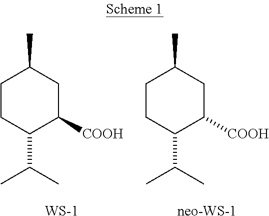

### BACKGROUND OF THE INVENTION

Acids such as isomeric p-menthane-3-carboxylic acids, including WS-1 and neo-WS-1 (Scheme 1) are key intermediates in the syntheses of certain biologically active materials, especially physiological cooling agents. Numerous examples of such syntheses can be found in Erman, Perfumer & Flavorist 2007, V 32, pp. 20-35; Leffingwell, Cooling Ingredients and Their Mechanism of Action in Handbook of Cosmetic Science and Technology, 3^{rd} Ed., A. O. Barel, M. Paye, H. I. Mailbach, Eds., Informs Healthcare, N.-Y., 2009, pp. 661-675, and Yelm at al. in U.S. Pat. Appl. 2010/0076080.

A known and widely-used process for making WS-1 is described in GB 1,392,907, and consists of a reaction of menthyl chloride with Mg to form a Grignard reagent—menthyl magnesium chloride—and carbonation of the Grignard reagent with CO₂ followed by hydrolysis (Scheme 2). The method produces practically pure “normal” WS-1 with an equatorial configuration of the carboxylic group.

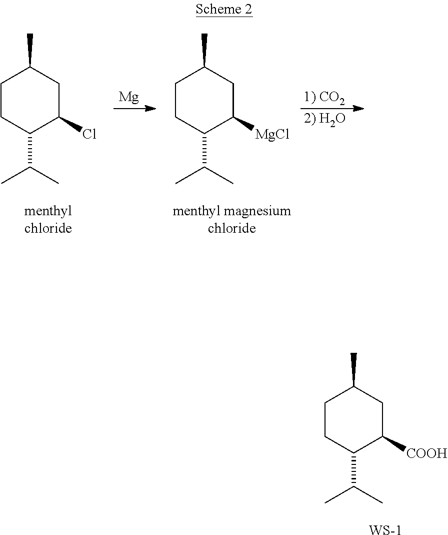

In a method of making neo-WS-1 suggested by Diliner, Organic Preparations and Procedures International 2009, V 41, pp. 147-152, I-menthol is converted in its mesylate, which reacts with Na cyanide to give neo-WS-1 nitrile. Next, the nitrile is treated with organo aluminum reagent DIBAL-H providing neo-WS-1 aldehyde, which is then oxidized into neo-WS-1 using Jones reagent CrO₃/H₂SO₄/acetone (Scheme 3).

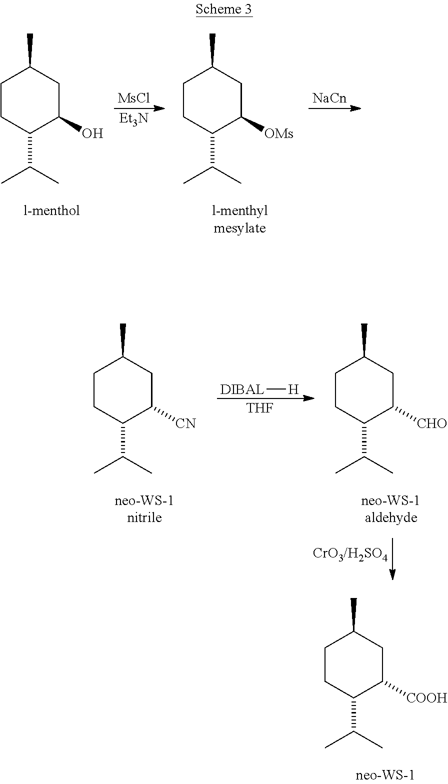

Co-pending U.S. patent application Ser. No. 12/940,063 by Erman et al. teaches a novel process that provides mixtures of neo-WS-1 and WS-1, significantly enriched in the neo-isomer. The process (Scheme 4) consists of contacting an oxaspiro compound with catalytic amount of a Lewis acid to make a mixture of neo- and normal WS-1 aldehydes in a ratio about 2:1, which mixture is aerobically oxidized into a mixture of neo-WS-1 and WS-1 in about same ratio 2:1. The application is silent about a possibility of obtaining pure neo-WS-1 and/or “normal” WS-1 by separation of the mixture. It is very hard, if not impossible, to predict whether separation of these two structurally close compounds would be possible and/or economical on industrial scale.

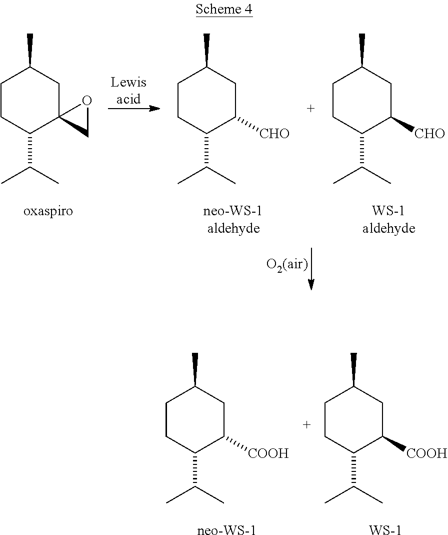

As shown above, known approaches to WS-1, neo-WS-1 and their mixtures are chemically quite different. A manufacturer, who would like to produce both isomers in a pure form, would have to implement two different product lines: Grignard-based for WS-1 and Cyanation-based for neo-WS-1. Therefore, it would be beneficial to find a method of interconversion between WS-1 and neo-WS-1.

There is no information on direct interconversions between WS-1 and neo-WS-1 in the literature. By analogy, what could be considered prior art for such interconversions is U.S. Pat. No. 5,831,118, which teaches epimerization of cis-isomers (or their mixtures with some trans-isomer) of potassium salts of 4- or 2-alkyl substituted cyclohexanecarboxylic acids at 130° C.-220° C. into practically pure trans-isomers. In Examples 1-4 of U.S. Pat. No. 5,831,118, a mixture of acid isomers (predominantly cis) in a solvent is converted into K-salts using two-fold excess of KOH, heated and then converted into trans-acid by reaction with excess HCl. Drawbacks of this approach is the necessity of converting acid into the salt using excess KOH, then the necessity of recovery of the product acid using again an excess of HCl.

Therefore, a need still exists for a method that would allow a catalytic direct interconversion between WS-1 and neo-WS-1.

### SUMMARY OF THE INVENTION

One embodiment relates to a process for interconversion between WS-1 and neo-WS-1. The process can include exposing a reaction mixture to a temperature greater than or equal to 60 degrees Celsius. The reaction mixture can include WS-1, neo-WS-1, or mixtures thereof. For example, the starting composition can include greater than or equal to 98% by weight WS-1, greater than or equal to 98% by weight neo-WS-1, or a mixture of WS-1 and neo-WS-1 in any ratio. The reaction mixture can further include an acid catalyst.

The process can produce a product comprising greater than or equal to 98% by weight neo-WS-1 or greater than or equal to 98% by weight WS-1. WS-1 can be isolated from the product by a method selected from the group consisting of distillation, crystallization, and combinations thereof. Neo-WS-1 can be removed from the product by distillation. The process can produce a product containing WS-1 and neo-WS-1 in a ratio respectively of 7.3±1.0.

Another embodiment relates to a method that includes heating a starting composition comprising greater than or equal to 98% by weight WS-1 in a reaction zone to a temperature of from 60 degrees Celsius to 250 degrees Celsius, and removing neo-WS-1 from the reaction zone by distillation to obtain a product composition comprising greater than or equal to 98% by weight neo-WS-1. The heating can be conducted in the presence of an acid catalyst.

Another embodiment relates to a method that includes heating a starting composition comprising a mixture of neo-WS-1 and WS-1 in any ratio to a temperature of from 60 degrees Celsius to 250 degrees Celsius to make an intermediate composition; and isolating WS-1 from the intermediate composition by a method selected from the group consisting of distillation, crystallization, and combinations thereof to obtain a product composition comprising greater than or equal to 98% by weight WS-1.

These and other features, aspects, and advantages will become better understood with reference to the following description and appended claims.

### DETAILED DESCRIPTION OF THE INVENTION

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure. Numerical ranges include all values within the range. For example, a range of from 1 to 10 supports, discloses, and includes the range of from 5 to 9. Similarly, a range of at least 10 supports, discloses, and includes the range of at least 15. Unless otherwise specified all amounts are expressed as weight percentages.

According to various embodiments WS-1 and neo-WS-1 can be interconverted at elevated temperatures. Regardless of the initial ratio of WS-1 to neo-WS-1, or even starting from pure WS-1 or pure neo-WS-1, the reaction comes to equilibrium at the ratio WS-1/neo-WS-1-7.3±1.0 (Scheme 5). The process can be significantly accelerated in the presence of an acid catalyst, where the catalyst can be a Brφnsted or a Lewis acid.

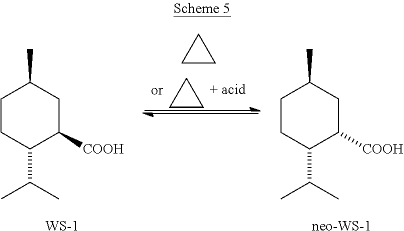

The process can be run in a batch mode or continuously, in the presence of a solvent, or preferably without a solvent. The solvent can be selected from aliphatic hydrocarbons such as heptane, octane, nonane, decane, undecane, dodecahe, tridecane, tetradecane, pentadecane, hexadecane and their isomers and mixtures thereof; aromatic hydrocarbons such as toluene, xylenes, cumene, cymene and mixtures thereof, ethers such as dibutyl ether and diphenyl ether, and esters such as isopropyl myristate.

The interconversion of WS-1 and neo-WS-1 can be conducted at a temperature within a range having a lower limit and/or an upper limit, each expressed degrees Celsius. The range can include or exclude the lower limit and/or the upper limit. The temperature lower limit and/or upper limit can be selected from 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, and 250 degrees Celsius. For example, the temperature can be in the range of greater than 60 degrees Celsius, less than 250 degrees Celsius, 60 degrees Celsius to 250 degrees Celsius, or 150 degrees Celsius to 220 degrees Celsius.

As stated above, the process can be significantly accelerated in the presence of an acid catalyst, where the catalyst can be a Brφnsted or a Lewis acid. A Brφnsted acid can include, but is not limited to, mineral acids and organic acids. Examples of mineral Brφnsted acids include, but are not limited to: sulfuric, phosphoric, perchloric, and the like. Examples of organic Brφnsted acids include, but are not limited to: p-toluenesulfonic, sulfosalycilic, benzenesulfonic, methanesulfonic, triflic, and the like. A Lewis acid can include, but is not limited to, all metal cations, and electron-deficient molecules such as boron trifluoride and trichloride, aluminum trichloride, titanium tetrachloride, antimony pentafluoride, and the like. Examples of Lewis acids include, but are not limited to: zinc bromide, zinc chloride, boron trifluoride, ferric chloride, and lithium perchlorate.

In another embodiment of the present invention, starting from practically pure (=98%) WS-1, practically pure (=98%) neo-WS-1 is obtained by heating WS-1, to a temperature defined above, in the absence or in the presence of a catalytic amount of an acid and shifting the equilibrium towards neo-WS-1 by removing it, as it forms, from the reaction zone by distillation. Using the same technique, practically pure (=98%) neo-WS-1 can be obtained starting from mixtures of WS-1 and neo-WS-1 containing them in any ratio.

In another embodiment of the invention, heating practically pure (=98%) neo-WS-1 or a mixture of neo-WS-1 and WS-1 in any ratio, to a temperature defined above, in the absence, or preferably in the presence of a catalytic amount of an acid results in reaching the equilibrium, where WS-1 is predominant. After removal of the acid catalyst by aqueous wash or neutralization, the enriched WS-1 can be purified to =98% by crystallization from a co-melt or from a solution in an appropriate solvent, for example, pentane, hexane, heptane, benzene, toluene, methyl acetate, ethylacetate, or the like. Alternatively, it can be purified by distillation. Using the same technique, practically pure (=98%) WS-1 can be obtained starting from mixtures of WS-1 and neo-WS-1 containing them in any ratio.

Thus, the proposed invention provides a simple and efficient catalytic method for interconversions between WS-1 and neo-WS-1 and provides easy access to pure WS-1 and neo-WS-1.

The following examples are given only for illustration of the invention. Those skilled in the art will recognize numerous variations that are within the spirit of the invention and scope of the claims.

### EXAMPLES

### Example 1

### Thermal Isomerization of WS-1 at 220° C.

WS-1 (60.0 g, purity 98.0%) is stirred at 220° C. under nitrogen and the mixture is periodically analyzed by GC. In 25 hours, the mixture contains 94.5% of WS-1 and 3.8% of neo-WS-1. In 74 hours, the mixture contains 86.1% of WS-1 and 11.9% of neo-WS-1 (ratio WS-1/neo-WS-1=7.25).

### Example 2

### Thermal Isomerization of WS-1 at 250° C.

WS-1 (60.0 g, purity 98.0%) is stirred at 250° C. under nitrogen and the mixture is periodically analyzed by GC. In 10 hours, the mixture contained 78.4% of WS-1 and 10.1% of neo-WS-1 (ratio WS-1/neo-WS-1=7.76).

### Example 3

### Thermal Isomerization of neo-WS-1 at 175° C.

Neo-WS-1 (60 g, purity 98.0%) is stirred at 175° C. under nitrogen and the mixture is periodically analyzed by GC. In 72 hours, the mixture contains 86.5% of WS-1 and 11.2% of neo-WS-1 (ratio WS-1/neo-WS-1=7.72).

### Example 4

### Obtaining Pure WS-1 by Acid Catalyzed Isomerization of a Mixture of WS-1 and Neo-WS-1

A solution of 1.4 g of p-toluenesulfonic acid monohydrate in a mixture of WS-1 and neo-WS-1 (95.9 g, ratio WS-1/neo-WS-1˜0.48) is stirred at 175° C. under nitrogen, and the mixture is periodically analyzed by GC. In 32 hours, the mixture contains 84.1% of WS-1 and 11.4% of neo-WS-1 (ratio WS-1/neo-WS-1=7.38). After addition of 150 ml of heptane, the mixture is washed with water, the organic layer is separated, filtered through a pad of anhydrous Na sulfate, slowly cooled to −5° C., and the crystals of WS-1 are quickly filtered off and dried on filter (63.3 g, purity 93.0%). Recrystallization from heptane affords 55.6 g of 98.0% pure WS-1.

### Example 5

### Acid Catalyzed Isomerization of Neo-WS-1 at 175° C.

A mixture of neo-WS-1 (106.4 g, purity 98.0%) and 1.9 g of p-toluenesulfonic acid monohydrate is stirred at 175° C. under nitrogen and periodically analyzed by GC. In 23 hours, the mixture contains 85.9% of WS-1 and 11.75% of neo-WS-1 (ratio WS-1/neo-WS-1=7.31).

### Example 6

### Acid Catalyzed Isomerization of WS-1 at 175° C.

A mixture of WS-1 (106.3 g, purity 98.0%) and 1.9 g of p-toluenesulfonic acid monohydrate is stirred at 175° C. under nitrogen and periodically analyzed by GC. In 15 hours, the mixture contains 86.6% of WS-1 and 11.2% of neo-WS-1 (ratio WS-1/neo-WS-1=7.73).

### Example 7

### Acid Catalyzed Isomerization of Neo-WS-1 at 175° C.

A mixture of neo-WS-1 (60 g, purity 98.0%) and 2.4 g of sulfosalycilic acid dihydrate is stirred at 175° C. under nitrogen and periodically analyzed by GC. In 24 hours, the mixture contains 85.6% of WS-1 and 11.6% of neo-WS-1 (ratio WS-1/neo-WS-1=7.38).

### Example 8

### Lewis Acid Catalyzed Isomerization of Neo-WS-1 at 175° C.

A mixture of neo-WS-1 (60 g, purity 98.0%) and 6.6 g of zinc bromide is stirred at 175° C. under nitrogen and periodically analyzed by GC. In 30 hours, the mixture contains 84.7% of WS-1 and 12.0% of neo-WS-1 (ratio WS-1/neo-WS-1=7.06).

### Example 9

### Lewis Acid Catalyzed Isomerization of Neo-WS-1 at 60° C.

A mixture of neo-WS-1 (60 g, purity 98.0%) and 2.0 g of boron trifluoride etherate is stirred at 60° C. under nitrogen and periodically analyzed by GC. In 30 hours, the mixture contains 77.2% of WS-1 and 11.0% of neo-WS-1 (ratio WS-1/neo-WS-1=7.02).

### Example 10

### Obtaining Pure Neo-Ws-1 from WS-1 by Catalytic Reactive Distillation

WS-1 (2400 g) and 43.8 g of p-toluenesulfonic acid monohydrate is charged to a 5-liter flask equipped with a magnetic stirrer, electrical heating mantel and 41×1″ distillation column filled with stainless steel packing “Pro Pak®.” The mixture is heated, stirred and slowly distilled overhead at ˜1 mm Hg. Distillation parameters and results are given in Table 1.

TABLE 1

| | | Top column | Stillpot | | |
|---|---|---|---|---|---|
| | Cut | temperature, | temperature, | % Neo WS-1 | |
| Cut | wt, g | ° C. | ° C. | (GC) | % WS-1 (GC) |
| 1 | 214.4 | 108 | 177-180 | 68.29 | 23.08 |
| 2 | 213.3 | 108 | 180-176 | 74.87 | 20 |
| 3 | 210.8 | 108-105 | 176-173 | 76.52 | 19.84 |
| 4 | 212.1 | 105-102 | 173-175 | 85.52 | 12.83 |
| 5 | 211.7 | 102 | 175-173 | 92.84 | 6.18 |
| 6 | 214.2 | 102 | 173-174 | 93.07 | 6.22 |
| 7 | 231.2 | 102-104 | 174-176 | 94.32 | 5.08 |
| 8 | 195.6 | 104-111 | 176-178 | 92.78 | 5.87 |
| 9 | 203.9 | 111-110 | 178-175 | 93.01 | 6.28 |
| 10 | 199.7 | 110-115 | 175-156 | 96.28 | 2.72 |
| 11 | 39.2 | 115-97 | 156 | 93.65 | 3.64 |

Cuts 5-11 are combined and redistilled in the same column at about 0.5 mm Hg, but without p-toluenesulfonic acid catalyst to give 782.3 g of 98.0% pure neo-WS-1 (first pass yield 32.6%). All other cuts from both distillations are mixtures of WS-1 and neo-WS-1 in various proportions and are reprocessed similarly through catalytic reactive distillation and redistillation to give additional pure neo-WS-1.

Although the present invention has been described in considerable detail with reference to certain preferred versions thereof, other versions are possible. Therefore, the spirit and scope of the appended claims should not be limited to the description of the preferred versions contained herein.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. §112, sixth paragraph. In particular, the use of “step of” in the claims herein is not intended to invoke the provisions of 35 U.S.C. §112, sixth paragraph.

What is claimed is:

1. A process for interconversion between WS-1 and neo-WS-1, the process comprising exposing a reaction mixture comprising a starting composition selected from the group consisting of WS-1, neo-WS-1, and mixtures thereof, to a temperature greater than or equal to 60 degrees Celsius.

2. The process of claim 1, wherein the temperature is in a range of from 60 degrees Celsius to 250 degrees Celsius.

3. The process of claim 1, wherein the temperature is in a range of from 150 degrees Celsius to 220 degrees Celsius.

4. The process of claim 1, wherein the reaction mixture further comprises an acid catalyst.

5. The process of claim 4, wherein the acid catalyst is a Brφnsted acid.

6. The process of claim 5, wherein the Brφnsted acid is selected from the group consisting of p-toluenesulfonic, sulfosalycilic, benzenesulfonic, methanesulfonic, triflic, sulfuric, phosphoric, perchloric, and combinations thereof.

7. The process of claim 4, wherein the acid catalyst is a Lewis acid.

8. The process of claim 7, wherein the Lewis acid is selected from the group consisting of zinc bromide, zinc chloride, boron trifluoride, ferric chloride, lithium perchlorate, and combinations thereof.

9. The process of claim 1, wherein the starting composition comprises greater than or equal to 98% by weight WS-1.

10. The process of claim 1, wherein the starting composition comprises greater than or equal to 98% by weight neo-WS-1.

11. The process of claim 1, wherein the starting composition comprises a mixture of WS-1 and neo-WS-1 in any ratio.

12. The process of claim 1, wherein the process produces a product comprising greater than or equal to 98% by weight neo-WS-1.

13. The process of claim 1, wherein the process produces a product comprising greater than or equal to 98% by weight WS-1.

14. The process of claim 1, wherein the process produces a product, and the process further comprises isolating WS-1 from the product by a method selected from the group consisting of distillation, crystallization, and combinations thereof.

15. The process of claim 1, wherein the process produces a product, and the process further comprises continuously removing neo-WS-1 from the product by distillation.

16. The process of claim 1, wherein the process produces a product containing WS-1 and neo-WS-1 in a ratio respectively of 7.3±1.0.

17. A method comprising
heating a starting composition comprising greater than or equal to 98% by weight WS-1 in a reaction zone to a temperature of from 60 degrees Celsius to 250 degrees Celsius, to convert at least a portion of the WS-1 to neo-WS-1; and
removing neo-WS-1 from the reaction zone by distillation to obtain a product composition comprising greater than or equal to 98% by weight neo-WS-1.

18. The method of claim 17, wherein the heating is conducted in the presence of an acid catalyst.

19. A method comprising
heating a starting composition comprising a mixture of neo-WS-1 and WS-1 in any ratio to a temperature of from 60 degrees Celsius to 250 degrees Celsius to make an intermediate composition;
isolating WS-1 from the intermediate composition by a method selected from the group consisting of distillation, crystallization, and combinations thereof to obtain a product composition comprising greater than or equal to 98% by weight WS-1.

20. The method of claim 19, wherein the heating is conducted in the presence of an acid catalyst
